(19) United States
(12) Patent Application Publication
(10) Pub. No.: US 2025/0213823 A1
(43) Pub. Date: Jul. 3, 2025

(54) CONDUIT SECUREMENT DEVICE COMPRISING A FORCE SENSOR

(71) Applicant: JAVELO HEALTH LIMITED, Edinburgh (GB)

(72) Inventor: Ashton BARNETT-VANES, Edinburgh (GB)

(21) Appl. No.: 18/847,364

(22) PCT Filed: Mar. 21, 2023
(86) PCT No.: PCT/GB2023/050712

§ 371 Date: Sep. 16, 2024

(30) Foreign Application Priority Data
Mar. 21, 2022 (GB) 2203952.3

(51) Int. Cl. A61M 25/02

(52) U.S. Cl. CPC A61M 25/02; A61M 2025/0206; A61M 2205/0294; A61M 2205/18; A61M 2205/332; A61M 2205/3344; A61M 2230/63

(57) ABSTRACT

A conduit securement device (50) comprising a support portion (52), at least one elastically extendable first strap (54) coupled to the support portion (52) and comprising a portion (54) configured to attach around a first region of a conduit, at least one force sensor (74) attached to a sensor portion (68) of the device (50) and configured to sense a force applied to the device (50) as a result of a force applied to the conduit, produce a force signal representative of the force applied to the device, determine when the force signal at least meets one or more thresholds and generate an alarm signal, and an alarm in communication with the force sensor (74) and configured to receive the alarm signal and issue an alarm. A medical system is also provided comprising a conduit securement device and at least one conduit secured to the device.

### FIELD OF THE INVENTION

The invention relates to a conduit securement device, particularly, but not exclusively, for securing a conduit to a patient in a clinical setting, the device having a force sensor for sensing force applied to the device.

### BACKGROUND TO THE INVENTION

Medical treatments commonly involve infusing fluids into or withdrawing fluids from a patient, by connecting a conduit, such as a cannula, a needle, a fluid line, to a body cavity of the patient such as the thorax, abdomen, cranium, vein, artery, spinal cord, urethra, bowel etc. For example, an intravenous (IV) fluid line may be used to deliver drugs, deliver or exchange plasma or deliver hydration fluids to a patient, whereas bodily fluids may be removed via urinary or enteric catheterisation, or surgical drains may be required during or after surgery.

A patient or body part can remain relatively mobile while a conduit comprising a fluid line is attached and a patient may forget or be unaware of the fluid line, particularly for lengthy treatments or when regaining consciousness. A clinical setting may also be crowded with additional fluid lines and medical apparatus and clinicians may need to frequently work in close proximity to fluid lines. Accordingly, unplanned removal of lines from a patient is a common problem and can lower the quality of care and may lead to adverse clinical outcomes.

Adhesive or tape can be used to secure a conduit to a patient. This approach can be readily adapted to different types or diameters of conduit. However, hair, sweat or other fluids can weaken the adhesion of the conduit to a patient's skin and at best only limited conduit pull resistance can be achieved. Adhesive tape can also be uncomfortable for the patient.

Dialysis lines (amongst many others) are a particular use case for conduits in a clinical setting. Dislodgement of such lines cause significant patient morbidity and mortality. Patients are increasingly dialysing at home or overnight, remote from a hospital. This leads to better patient experience but magnifies the risks if a dialysis line dislodgement occurs. For example, if the patient rolls over, the dialysis line can be subject to a force and can dislodge.

The current market standard in dialysis is use of enuresis pads. These initiate a patient alert on becoming soaked by blood from the patient, after dislodgement of a dialysis line has taken place. This is not ideal and accordingly there remains a need to address one or more deficiencies encountered with existing conduit/fluid line securement and alerts to patients.

In other fields, there are circumstances in which a conduit is attached to a device and the application of force to, and possible dislodgement of, such conduits is undesirable. For example, an electrical signal cable may be attached to a monitoring device. Application of force to the cable and dislodgement thereof could result in disruption to or cessation of signals from the monitoring device. In such a circumstance, it is desirable for an operator of the monitoring device to be aware of force applied to the electrical cable.

### SUMMARY OF THE INVENTION

According to a first aspect of the invention there is provided a conduit securement device comprising:

a support portion,

at least one elastically extendable first strap coupled to the support portion and comprising a portion configured to attach around a first region of a conduit,

at least one force sensor attached to a sensor portion of the device and configured to sense a force applied to the device as a result of a force applied to the conduit, produce a force signal representative of the force applied to the device, determine when the force signal at least meets one or more thresholds and generate an alarm signal, and

an alarm in communication with the force sensor and configured to receive the alarm signal and issue an alarm.

The conduit may be a rigid conduit. The rigid conduit may be any of a cannula, a needle. The conduit may be a flexible conduit. The flexible conduit may be any of a fluid line, a dialysis line, a catheter, a drain, an electrical signal cable, an optical signal cable, a wire.

The first strap may be coupled to the support portion to hold the conduit at any orientation to the support portion.

The conduit securement device may further comprise an elastically extendable second strap coupled to the support portion and comprising a portion configured to attach around a second region of the flexible conduit, wherein the first strap and second strap are disposed on the support portion in linearly offset positions such that the first region of the flexible conduit is linearly offset from the second region of the flexible conduit and the flexible conduit is urged into a pathway across the device.

The second strap may be attached to the first strap. The first strap may be attached to the second strap. This couples the first strap to the support portion.

The first strap or second strap may be coupled to the support portion by any of a hinged structure, a living hinge, a hook and loop fastener, a snap-fit fastener, an eyelet and pin fastener, a hook and eyelet fastener, a button and eyelet fastener, a popper fastener, a hole and link fastener, a slider fastener, a clasp, a squeeze buckle, a D-ring fastener, a magnetic snap fastener, a cam buckle, a ratchet buckle, a slide buckle, a side release buckle, a tie buckle, a ratchet teeth and pawl fastener, a latch, a lever, a button for setting/releasing a latch.

The conduit securement device may further comprise two elastically extendable second straps coupled to the support portion each comprising a portion configured to attach respectively around second and third regions of the flexible conduit, wherein the first strap and the second straps are disposed on the support portion in linearly offset positions such that the first region of the flexible conduit is between and linearly offset from the second and third regions of the flexible conduit and the flexible conduit is urged into a curved or convoluted pathway across the device.

In use the portion of the first strap and the portions of the one or more second straps, when present, attach around (e.g. wrap around) a conduit to secure the conduit to the device. The static friction between the first strap, and the second straps when present, and the conduit provide a degree of resistance i.e. resistance to movement of the conduit in relation to the securement device. The pull resistance is enhanced by the curved or convoluted pathway, when present. The first strap and the second straps, when present, resist a force placed on one side of the conduit transmitting through to the other side of the conduit.

Force applied to the conduit longitudinally (i.e. wherein at least a component of force is applied along a length of the conduit), proximal to or distal from the securement device, acts to pull the conduit through the portion of the first strap. Force applied to the conduit longitudinally, proximal to or distal from the securement device, acts to pull the conduit through the or each portion of the one or more second straps, when present, and can try to straighten the pathway of the conduit. As the conduit is pulled, the first strap and the one or more second straps, when present, elastically extend (or further elastically extend), increasing the static friction with the conduit and resisting longitudinal displacement of the conduit. In effect, the securement device isolates the conduit extending distally from the device from forces applied between the securement device and the conduit extending proximally from the device, and vice versa. The securement device thereby reduces the risk that an end of the conduit attached to, for example a patient or an apparatus, such as a medical apparatus or any other type of apparatus, is disrupted. Furthermore, the elastically deformable straps can deform to accommodate lateral movement or bending of the conduit proximal or distal to the device, reducing the risk of bending or crimping of the conduit.

In some embodiments, all of the or each of the straps is elastically extendable. In other embodiments, at least a portion of the at least one first strap and at least a portion of the at least second strap, when present, are elastically extendable. The portions of the at least one first strap and the at least second strap, when present, which are elastically extendable may comprise loops which are configured to be attached to respectively the first region of the conduit, and the second region of the conduit and the third region of the conduit.

The conduit securement device may further comprise an external attachment for attaching the device to a patient or a structure such as a medical apparatus, a machine, a trolley, a bed, a dialysis apparatus, an infusion apparatus, an incubator. The external attachment may for example comprise a band. This may attach around a patient's body part, such as a limb, torso, shoulder, neck, foot, head, waist or around a part of a structure. The external attachment may comprise a releasable fastening, such as a clip or a component of a hook and loop fastening. The external attachment may comprise a garment, or garment portion, such as a vest, undergarment or limb covering. The external attachment may comprise an adhesive portion, for example in the form of a tape or an adhesive patch or strip. This may be applied to a patient's body part, such as a limb, torso, shoulder, neck, foot, head, waist, a patient's clothing, or to a structure such as a medical apparatus, a piece of furniture, a machine, an incubator or a trolley.

The force sensor may be configured to sense force applied to the device as a result of a force applied to the conduit arising from any of compressing, pulling, bending, moving, vibrating, elongating the conduit.

The force sensor may be configured to sense one or more of a strain force, a shear force, a pressure force, a dynamic force applied to the conduit securement device.

The force sensor may comprise one or more of a strain force sensor, a pressure force sensor, a dynamic force sensor, a rotation force sensor.

The force sensor may process the force signal to quantify the force. The force sensor may dynamically process the force signal to quantify the force. The force sensor may process the force signal in real time to quantify the force. The force sensor may process the force signal to determine a direction of the force. The force sensor may process the force signal to add one or more time stamps. The one or more time stamps may record and log any of treatment time events, adverse events. These may be seen and reviewed by care providers in real time or retrospectively. For example, for treatment or research or audit purposes.

The force sensor may process the force signal to determine when the force signal at least meets a force magnitude threshold and generate an alarm signal. The force sensor may process the force signal to determine when the force signal at least meets a force magnitude threshold and at least meets a force duration threshold and generate an alarm signal. The force magnitude threshold may be any of 10 N, 20 N, 30 N, 40 N, 60 N, 100 N, 140 N. The force magnitude threshold may be adjustable by a user of the conduit securement device. The force duration threshold may be any of 0.1 secs, 1 sec, 2 secs, 3 secs, 4 secs, 5 secs, 10 secs, 20 secs. The force duration threshold may be adjustable by a user of the conduit securement device. When the conduit is, for example, a fluid line for a patient, such durations of such force magnitudes on the securement device resulting from a force on the conduit may lead to eventual dislodgement of the fluid line conduit from the patient. The forces may also compromise flow through the conduit which should be avoided. The force sensor may process the force signal to monitor trends in the force signal. The force sensor may generate an alarm signal when a particular trend or trends are detected.

The force sensor may comprise a force sensing element and a signal processing unit in communication with the force sensing element. The force sensing element may sense a force applied to the conduit securement device, generate a force signal representative of the force and output the force signal to the signal processing unit. The signal processing unit may determine when the force signal at least meets one or more thresholds and generate an alarm signal.

The force sensing element may be positioned at the sensor portion of the conduit securement device comprising a first location of the conduit securement device and the signal processing unit may be positioned at a second location of the conduit securement device. The force sensing element may be placed in a first housing of the force sensor and the signal processing unit may be placed in a second housing of the force sensor. The force sensing element may be in wireless communication with the signal processing unit. The force sensing element may be in wired communication with the signal processing unit.

The force sensing element and the signal processing unit may be substantially collocated at the sensor portion of the conduit securement device. The force sensing element and the signal processing unit may be placed in a housing of the force sensor.

The signal processing unit may be separate from the conduit securement device. The signal processing unit may be provided in a computing device separate from the conduit securement device. The force sensing element may be in wireless communication with the signal processing unit. The force sensing element may be in wired communication with the signal processing unit. This allows processing of the force signal to be done remotely, for example on a computing device, such as a computer or phone, separate from the conduit securement device, such as at a care provider's computing device.

The force sensing element may be attached to the sensor portion of the conduit securement device. The force sensing element may be moulded within the sensor portion of the conduit securement device. The sensor portion of the conduit securement device may comprise any of a central section of the support portion of the device, an under surface of the support portion of the device, a portion of the first strap configured to attach around the first region of the conduit, a portion of one of the two or more second straps configured to attach around a region of the conduit, an attachment device of any of the one or more first straps or the two or more second straps configured to attach a strap to the support portion or a further strap.

The signal processing unit may comprise at least one processor. The processor may be configured to receive a force signal representative of a force applied to the conduit securement device from the force sensing element. The processor may be configured to receive a plurality of force signals representative of forces applied to the conduit securement device from the force sensing element. The plurality of force signals may be received at regular time intervals, for example at 10 Hz intervals.

The signal processing unit may comprise at least one memory unit. The memory unit may be configured to store force sensor data. The force sensor data may comprise any of one or more magnitudes of one or more force signals representative of forces applied to the conduit securement device, one or more instances at which a force signal at least meets at least one threshold, one or more instances at which an alarm signal is generated, magnitudes of force signals which at least meet at least one threshold, duration of force signals which at least meet at least one threshold. The memory unit may store one or more force magnitude thresholds. The memory unit may store one or more force duration thresholds. The memory unit may store one or more time stamps. The memory unit may store other information such as movement data.

The signal processing unit may comprise at least one transceiver. The transceiver may output force sensor data to one or more computing devices. The force sensor data may be output via one or more wired connections to the one or more computing devices, such as one or more USB connections. The force sensor data may be output via one or more wireless connections to the one or more computing devices, such as one or more Bluetooth connections and/or one or more Wi-Fi connections and/or one or more SMS connections. The force sensor data may be output via a combination of one or more wired connections and one or more wireless connections to the one or more computing devices.

The one or more computing devices may comprise a device located in proximity to a patient using the conduit securement device. Additionally or alternatively, the one or more computing devices may comprise a device located in a patient care facility and configured to receive force sensor data and generate an alert for care personnel that a force is being applied to the conduit. The force sensor data may be continuously received by care personnel. The force sensor data may be received on a monitoring screen or platform. The force sensor data may be accessible remotely e.g. via a website or smartphone application. Additionally or alternatively, the one or more computing devices may comprise a device located with one or more care personnel and configured to receive force sensor data and generate an alert for the care personnel that a force is being applied to the conduit. The device located with the one or more care personnel may comprise, for example, a smart phone, a tablet, a laptop, comprising software, such as application software, configured to receive the force sensor data and generate an alert for the care personnel, such as a notification, a text message, a phone call. Additionally or alternatively, the one or more computing devices may comprise a device connected to the conduit and configured to receive force sensor data and generate an alert for the device that a force is being applied to the conduit. The device may be configured to use the alert to activate changing or attenuating of flow of liquid to the conduit. The device may be, for example, an infusion pump and/or a dialysis machine and may be configured to activate slowing the rate of infusion/dialysis or stopping the infusion/dialysis. The one or more computing devices may be configured to display force sensor data, for example using SerialPlot.

The force sensor may comprise at least one power source. The power source may be a battery. The power source may be thermal energy from a patient wearing the conduit securement device.

The alarm may comprise an audible alarm element configured to receive the alarm signal and issue an audible alarm. The audible alarm element may be a piezo sounder. Additionally or alternatively, the alarm may comprise a visual alarm element configured to receive the alarm signal and issue a visual alarm. The visual alarm element may be configured to use various colours and/or durations of visual alarms to indicate different alarm states of the alarm. The visual alarm element may be configured to issue a visual alarm comprising an SMS. The visual alarm element may be a RGB LED. The alarm may comprise an audio-visual alarm element configured to receive the alarm signal and issue an audio-visual alarm. The audio-visual alarm element may comprise a display screen. Additionally or alternatively, the alarm may comprise a haptic alarm element configured to receive the alarm signal and issue a haptic alarm.

The alarm may comprise an alarm cancellation device which, on activation, cancels the alarm. The alarm cancellation device may comprise a push button which, on pressing, cancels the alarm. Thus, if an alarm is triggered, a patient using the conduit securement device can change position to alleviate a force on the conduit and then cancel the alarm. The alarm may comprise an alarm interaction device configured to permit a patient using the conduit securement device to generate an alarm.

The force sensor may comprise a strain sensor configured to sense a strain force applied to the conduit securement device, produce a strain signal representative of the strain force, determine when the strain signal at least meets one or more thresholds and generate an alarm signal.

The strain sensor may process the strain signal to determine when the force signal at least meets a magnitude strain threshold and generate an alarm signal. The strain sensor may process the strain signal to determine when the strain signal at least meets a magnitude strain threshold and at least meets a duration threshold and generate an alarm signal.

The strain sensor may comprise a strain sensing element and a signal processing unit in communication with the strain sensing element. The strain sensing element may sense a strain applied to the conduit securement device, generate a strain signal representative of the strain and output the strain signal to the signal processing unit. The signal processing unit may determine when the strain signal meets one or more thresholds and generate an alarm signal.

The strain sensing element may comprise a strain gauge. The strain gauge may comprise a strain-sensitive cord. The strain gauge may comprise a printed mesh. The strain gauge may be placed on a base of the strain sensor. The base may be made of a flexible sheet material. The flexible material may be more flexible than a thin metal sheet and less flexible than a silicone sheet. The base may provide strain relief to protect the strain gauge.

The strain sensing element may comprise a strain-sensitive material which senses a strain force by a change of electrical state of the material. The strain-sensitive material may sense a strain force on flexing, for example stretching, of the material. The strain-sensitive material may be in the form of one or more strips of material. The strain-sensitive material may comprise a carbon-impregnated polyethylene strain-sensitive material. An example of a carbon-impregnated polyethylene strain-sensitive material is a Velostat (RTM) strain-sensitive material.

The strain sensing element may be attached to or moulded within a sensor portion of the conduit securement device comprising a central section of the support portion of the device. This section of the device deforms when force is applied to a conduit secured to the device. This section of the device is relatively fixed and is not subjected to repeated over bending when connecting and disconnecting the first strap and second straps, when present. In this section of the device, the strain sensing element should be unaffected by the size of the conduit.

The strain sensing element may be attached to or moulded within a sensor portion of the conduit securement device comprising an under surface of the support portion of the device.

In one embodiment, the strain sensing element may be attached to or moulded within a sensor portion of the conduit securement device comprising a portion of the first strap configured to attach around the first region of the conduit. In one embodiment, the strain sensing element may be attached to or moulded within a sensor portion of the conduit securement device comprising a portion of one of the two or more second straps configured to attach around a region of the conduit. This portion of the device will be a better position for a strain gauge strain sensing element which could be very sensitive. In one embodiment, the strain sensing element may be attached to or moulded within a sensor portion of the conduit securement device comprising a post of any of the one or more first straps or the two or more second straps.

The force sensor may comprise a pressure sensor configured to sense a pressure force applied to the conduit securement device, produce a pressure signal representative of the pressure force, determine when the pressure signal at least meets one or more thresholds and generate an alarm signal.

The pressure sensor may process the pressure signal to determine when the pressure signal at least meets a magnitude pressure threshold and generate an alarm signal. The pressure sensor may process the pressure signal to determine when the pressure signal at least meets a magnitude pressure threshold and at least meets a duration threshold and generate an alarm signal.

The pressure sensor may comprise a pressure sensing element and a signal processing unit in communication with the pressure sensing element. The pressure sensing element may sense a pressure force applied to the conduit securement device, generate a pressure signal representative of the pressure force and output the pressure signal to the signal processing unit. The signal processing unit may determine when the pressure signal at least meets one or more thresholds and generate an alarm signal.

The pressure sensing element may comprise a pressure-sensitive material which senses a pressure force by a change of electrical state of the material. The pressure sensing element may comprise a pressure-sensitive conductive material. The pressure sensing element may comprise a pressure-sensitive conductive fabric material. The pressure sensing element may comprise a carbon-impregnated polyethylene pressure-sensitive conductive material. An example of a carbon-impregnated polyethylene pressure-sensitive conductive material is a Velostat® carbon-impregnated polyethylene pressure-sensitive conductive material. The pressure sensing element may comprise one or more sheets of pressure-sensitive material. The pressure sensing element may comprise one or more strips of pressure-sensitive material.

The pressure-sensitive material may comprise a pressure-sensitive rubber material. The rubber pressure-sensitive material may be in the form of one or more strips of material or one or more pills of material.

The pressure-sensitive material may comprise a pressure-sensitive quantum tunnelling composite (QTC) material. The pressure-sensitive QTC material may be in the form of a membrane.

The pressure sensing element may be attached to or moulded within a sensor portion of the conduit securement device comprising a portion of the first strap configured to attach around the first region of a conduit. The pressure sensing element may be attached to or moulded within an inner surface of the portion of the first strap. The pressure sensing element may be attached to or moulded within an inner surface of the portion of the first strap to wrap around the first region of a conduit. When a force is applied to the conduit, for example by pulling the conduit, a pressure force is applied to the pressure sensing element.

The pressure sensing element may be attached to or moulded within a sensor portion of the conduit securement device comprising a portion of one or more of the second straps configured to attach around a region of a conduit. The pressure sensing element may be attached to or moulded within an inner surface of the portion of the one or more second straps. The pressure sensing element may be attached to or moulded within an inner surface of the portion of the one or more second straps to wrap around the region of a conduit.

The pressure sensing element may be attached to or moulded within a sensor portion of the conduit securement device comprising an under surface of the support portion of the device. The pressure sensing element may be attached to or moulded within a sensor portion of the conduit securement device comprising a post of any of the first strap portion or the two or more second straps.

The force sensor may comprise a composite force sensor configured to sense a pressure force and a strain force applied to the conduit securement device. The composite force sensor may produce a pressure force signal representative of the pressure force and a strain force signal representative of the strain force, determine when either or both of the pressure force signal and the strain force signal at least meet one or more thresholds and generate an alarm signal. The composite force sensor may comprise a pressure-sensitive conductive material to sense the pressure force. The composite force sensor may comprise a strain gauge to sense the strain force. The strain gauge may be mounted on the pressure-sensitive conductive material.

The force sensor may comprise a dynamic force sensor configured to sense a dynamic force applied to the conduit securement device, produce a dynamic force signal representative of the dynamic force, determine when the dynamic force signal at least meets one or more thresholds and generate an alarm signal.

The dynamic force applied to the conduit securement device may result from dynamic movements of the conduit, such as pulls, bending, cantilevering of the conduit, which can be in any direction. The dynamic movements of the conduit may result from, for example, short tugs of the conduit caused by catching on clothes etc.

The dynamic force sensor may process the dynamic force signal to determine when the dynamic force signal at least meets a magnitude dynamic threshold and generate an alarm signal.

The dynamic force sensor may comprise a dynamic force sensing element and a signal processing unit in communication with the dynamic force sensing element. The dynamic force sensing element may sense a dynamic force applied to the conduit securement device, generate a dynamic force signal representative of the dynamic force and output the dynamic force signal to the signal processing unit. The signal processing unit may determine when the dynamic force signal at least meets one or more thresholds and generate an alarm signal.

The dynamic force sensing element may comprise a piezoelectric material. The piezoelectric material may be in the form of a ribbon of piezoelectric material. The piezoelectric material may comprise a piezoresistive material. The piezoresistive material may provide a cantilever-type movement sensing element.

The dynamic force sensing element may be attached to a sensor portion of the conduit securement device comprising a portion of the one or more first strap configured to attach around a region of a conduit. The dynamic force sensing element may be attached to an inner surface of the portion of the one or more first straps.

The dynamic force sensing element may be attached to a sensor portion of the conduit securement device comprising a portion of one or more of the second straps configured to attach around a region of a conduit. The dynamic force sensing element may be attached to an inner surface of the portion of the one or more second straps.

The dynamic force sensor may be used with one or more other force sensors, such as a strain force sensor and/or a pressure force sensor.

The force sensor may comprise a rotation force sensor to sense a rotation caused by a force applied to one or more parts of the conduit securement device. The rotation force sensor may sense a rotation caused by a force applied to the first strap, when the conduit is subject to a force. The rotation of the first strap may take place around a post by which the first strap is coupled to the support structure of the conduit securement device.

The rotation force sensor may comprise a rotation sensing element and a signal processing unit in communication with the rotation sensing element. The rotation sensing element may sense a rotation caused by a force applied to one or more parts of the conduit securement device, generate a rotation signal representative of the rotation force and output the rotation signal to the signal processing unit. The signal processing unit may determine when the rotation signal at least meets one or more thresholds and generate an alarm signal.

The rotation sensing element may comprise a capacitor. The capacitor may comprise at least one first conductive plate and at least one second conductive plate separated by a dielectric, such as air, and/or one or more insulators. In one embodiment, the at least one first conductive plate and/or the at least one second conductive plate may be formed of any of a conductive ribbon, a conductive foil, a conductive fabric. The at least one first conductive plate may be attached to a portion of the support structure beneath the first strap and the at least one second conductive plate may be attached to the first strap.

In another embodiment, the at least one first conductive plate and/or the at least one second conductive plate may be formed of any of a carbon-impregnated elastomeric material, a silicone material, a carbon-impregnated ink. The at least one first conductive plate may be embedded in a portion of the support structure beneath the first strap and the at least one second conductive plate may be embedded in the first strap.

The capacitor may comprise a variable capacitor. The variable capacitor may comprise a first conductive plate located on a portion of the support section beneath the first strap and a plurality of second conductive plates of increasing area, connected together and located on an underside of the first strap. The plurality of second conductive plates may comprise any of 3 conductive plates, 4 conductive plates.

The variable capacitor may comprise a variable overlapping area between the first conductive plate and the plurality of second conductive plates. The variable capacitor may have a variable capacitance which is proportional to the variable overlapping area between the first conductive plate and the plurality of second conductive plates. The variable overlapping area between the first conductive plate and the plurality of second conductive plates may change with rotation angle of the first strap. The variable capacitor may have a variable capacitance which is proportional to rotation angle of the first strap.

The variable capacitor may be part of a relaxation oscillator connected to a microprocessor unit (MCU). The MCU may generate a signal and input the signal to the oscillator. The oscillator may output a frequency-modulated signal to the MCU. The MCU may measure the frequency of the frequency-modulated signal over a fixed window of time. The measured frequency may be inversely proportional to the capacitance of the variable capacitor and inversely proportional to rotation angle of the first strap.

The variable capacitor may be part of a RC circuit connected to a microprocessor unit. The MCU may generate a square-wave signal and input the square wave signal to the RC circuit. The RC circuit may output a modulated square-wave signal to the MCU. The MCU may use a rise time of modulated square waves of the modulated square-wave signal to determine capacitance of the variable capacitor and rotation angle of the first strap.

The rotation sensing element may comprise a first conductive plate having a plurality of isolated conductive strips and a second conductive plate. In one embodiment, the first conductive plate may be formed of alternate sections of conductive foil or fabric and the second conductive plate may be formed of a conductive ribbon or a conductive foil. The first conductive plate may be attached to a portion of the support structure beneath the first strap and the second conductive plate may be attached to the first strap and the position of the second conductive plate may change with rotation angle of the first strap. In another embodiment, the first conductive plate may be formed of alternate sections of conductive ink and the second conductive plate may be formed of a carbon-impregnated silicone. The first conductive plate may be embedded in a portion of the support structure beneath the first strap and the second conductive plate may be embedded in the first strap and the position of the second conductive plate may change with rotation angle of the first strap.

The first conductive plate having the plurality of isolated conductive strips may be connected to a MCU. The MCU may supply a voltage signal to each pair of adjacent conductive strips. Rotation of the first strap causes location of the second conductive strip adjacent at least one pair of adjacent conductive strips and a short circuit may be formed between the second conductive plate and the at least one pair of adjacent conductive strips. The MCU may detect the short circuit, determine the location of the second conductive strip and rotation of the first strap.

The rotation sensing element may comprise a zebra strip. The zebra strip may comprise an elastomeric material that has alternating conductive and insulating stripes which detects shorting of the stripes to determine rotation. The zebra strip may be attached to a sensor portion of the conduit securement device comprising a portion of the first strap. The zebra strip may be attached to an inner surface of the portion of the first strap.

The rotation force sensor may be used with one or more other force sensors, such as a strain force sensor and/or a pressure force sensor.

The force sensor may comprise a stretch sensor. The stretch sensor may be a capacitive stretch sensor. The stretch sensor may be a resistive stretch sensor. The stretch sensor may comprise a conductive material attached to or printed on or impregnated into a section of the conduit securement device, such as the first strap or one of the two or more second straps. The stretch sensor may be coupled with stretchable electronics.

The conduit securement device may further comprise an accelerometer. The accelerometer may sense general movement and vibration of the conduit securement device. The accelerometer may measure general movement of a patient wearing the conduit securement device. This may give an indication of patient activity and posture. One or more instances at which an alarm signal is generated may be correlated with one or more measurements from the accelerometer. This may be used to identify patient activity at the one or more instances to determine causes of the alarms and allow mitigation of the causes, for example by repositioning of the patient.

The conduit securement device may further comprise a sensor for monitoring contents passing through a conduit. The sensor for monitoring the contents passing through the conduit may comprise any of a detector in contact with the contents, a detector not in contact with the contents. The sensor for monitoring the contents passing through the conduit may comprise any of an ultrasound detector, an infra-red detector, a light detector, an electrical detector, a chemical detector. The sensor for monitoring the contents passing through the conduit may measure any of a flow rate, a flow volume, a colour, a biochemical property, bubbles in the contents, oxygen levels in the contents.

According to a second aspect of the invention there is provided a medical system comprising a conduit securement device according to the first aspect of the invention and at least one conduit secured to the device.

It will be understood that further and optional features of each aspect of the invention correspond to further and optional features of any other aspect of the invention.

### DESCRIPTION OF THE DRAWINGS

Example embodiments will now be described with reference to the following drawings in which:

FIG. 1 is a representation of a first embodiment of a conduit securement device according to the first aspect of the invention;

FIGS. 2a and 2b are representations of a second embodiment of a conduit securement device according to the first aspect of the invention in closed and open positions;

FIG. 3 is a representation of a third embodiment of a conduit securement device according to the first aspect of the invention;

FIG. 4 is a representation of a fourth embodiment of a conduit securement device according to the first aspect of the invention;

FIG. 5 is a representation of an embodiment of a conduit securement device according to the first aspect of the invention attached to an arm of a patient;

FIG. 6 is a representation of an embodiment of a conduit securement device according to the first aspect of the invention attached to a torso of a patient;

FIG. 7 is a representation of a first embodiment of a conduit securement device according to the first aspect of the invention attached to the neck of a patient;

FIG. 8 is a representation of a second embodiment of a conduit securement device according to the first aspect of the invention attached to the neck of a patient;

FIG. 9 is a representation of a strain sensor of a conduit securement device according to the first aspect of the invention;

FIG. 10 is a representation of a pressure sensor of a conduit securement device according to the first aspect of the invention, and

FIG. 11 is a representation of a rotation force sensor of a conduit securement device according to the first aspect of the invention.

### DETAILED DESCRIPTION OF EXAMPLE EMBODIMENTS

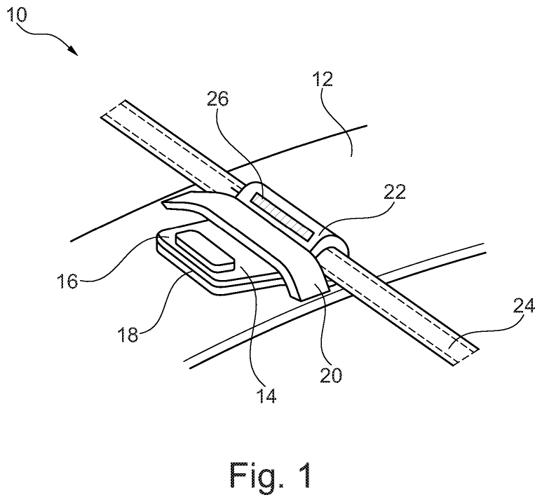

Referring to FIG. 1, a representation of a first embodiment of a conduit securement device 10 comprises a support portion 12 and an elastically extendable first strap 14. The first strap 14 is releasably coupled at free ends 16, 18 to the support portion 12 of the device 10 by a band 20 attached to the support portion 12.

A portion 22 of the first strap 14 between the free ends 16, 18 is configured to attach around a first region of a conduit 24. The portion 22 defines a channel running transversely across the support portion 12. The conduit 24 is placed in the channel and the portion 22 of the first strap 14 wraps around the first region of the conduit 24, to secure the conduit 24 to the conduit securement device 10.

When a force is applied between the conduit securement device 10 and the secured conduit 24, proximate or distal to the device 10, at least a component of which force acts along the conduit 24, this acts to try to pull the conduit 24 through the portion 22 of the first strap 14. This elastically extends, or further elastically extends, the first strap 14, which increases a static friction between the strap 14 and the conduit 24 and resists longitudinal displacement of the conduit 24 in relation to the securement device 10.

The conduit securement device 10 comprises a force sensor 26 attached to a sensor portion of the device 10 comprising the portion 22 of the first strap 14. The force sensor 26 is configured to sense a force applied to the device 10 as a result of a force applied to the conduit 24, produce a force signal representative of the force, determine when the force signal at least meets one or more thresholds and generate an alarm signal. The conduit securement device 10 further comprises an alarm (not shown) in communication with the force sensor and configured to receive the alarm signal and issue an alarm. The alarm may be placed within the force sensor 26.

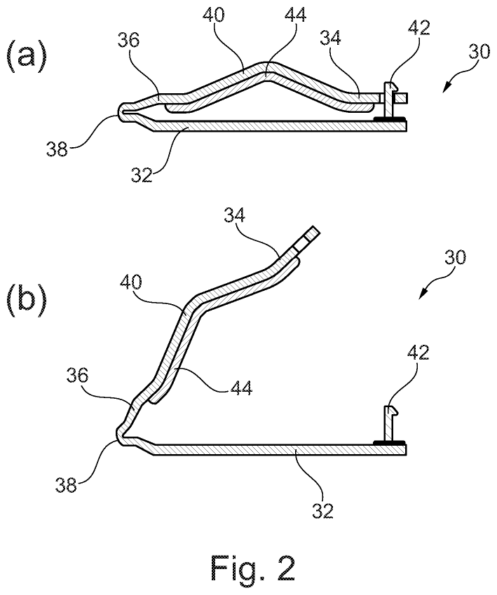

Referring to FIGS. 2a and 2b, a representation of a second embodiment of a conduit securement device 30 comprises a support portion 32 and an elastically extendable first strap 34. The first strap 34 is coupled at an end 36 to the support portion 32 of the device 30 by a hinge 38.

A portion 40 of the first strap 34 is configured to attach around a first region of a conduit (not shown). The portion 40 is substantially U-shaped and defines a channel running transversely across the support portion 32. The first strap 34 is pivoted about the hinge 38, away from the support portion 32, to allow the conduit to be placed in the channel of the portion 40 (FIG. 2b). The first strap 34, with the conduit, is then pivoted about the hinge 38, towards the support portion 32, to secure the conduit between the first strap 34 and the support portion 32 (FIG. 2a). The first strap 34 and the support portion 32 are fastened together with a releasable locking mechanism 42.

When a force is applied between the conduit securement device 30 and the secured conduit, proximate or distal to the device 30, at least a component of which force acts along the conduit, this acts to try to pull the conduit through the U-shaped portion 40 of the first strap 34. This elastically extends, or further elastically extends, the first strap 34, which increases a static friction between the strap 34 and the conduit and resists longitudinal displacement of the conduit in relation to the securement device 30.

The conduit securement device 30 comprises a force sensor 44 attached to a sensor portion of the device 30 comprising the portion 40 of the first strap 34. The force sensor 44 is configured to sense a force applied to the device 30 as a result of a force applied to the conduit, produce a force signal representative of the force, determine when the force signal at least meets one or more thresholds and generate an alarm signal. The conduit securement device 30 further comprises an alarm (not shown) in communication with the force sensor and configured to receive the alarm signal and issue an alarm.

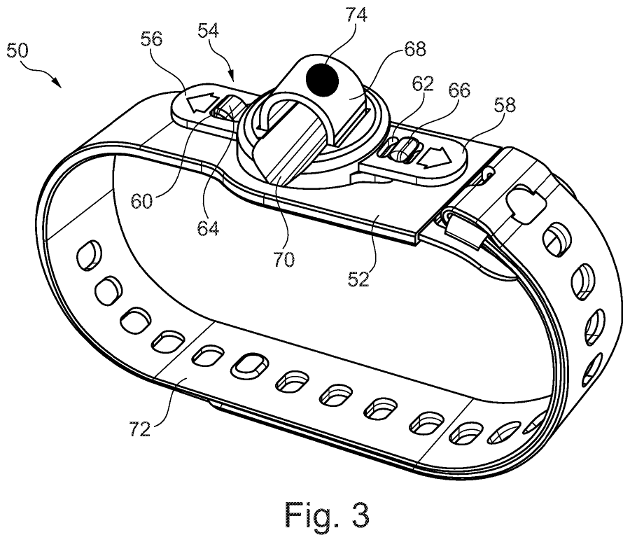

FIG. 3 shows a conduit securement device 50 which comprises a support portion 52 and an elastically extendable first strap 54. The first strap 54 is formed from an elastomeric material and is releasably attached at free ends 56, 58 to the support portion 52 of the device 50. Releasable attachment is effected by way of apertures 60, 62 through the free ends 56, 58, through which hooks 64, 66 are introduced. The hooks and apertures together form releasable fixings. It will be understood that alternative embodiments of the fixings may be used.

A portion 68 of the first strap 54 between the free ends 56, 58 is configured to form a loop which defines a channel 70 running transversely across the support portion 52. A conduit (not shown) is placed in the channel 70 and the portion 68 of the first strap 54 wraps around a region of the conduit, to secure the conduit to the conduit securement device 50.

The conduit securement device 50 further includes an external attachment comprising, in the embodiment shown, an adjustable wrist band 72 that extends from the support portion 52. This is used to attach the conduit securement device 50 to a patient or a structure.

When a force is applied between the conduit securement device 50 and parts of a secured conduit proximate or distal to the device 50, at least a component of which force acts along the conduit, and tries to pull the conduit through the loop portion 68 of the first strap 54. This elastically extends, or further elastically extends, the first strap 54, which increases a static friction between the strap and the conduit and resists longitudinal displacement of the conduit in relation to the securement device 50.

The conduit securement device 50 comprises a force sensor 74 attached to a sensor portion of the device 50 comprising the portion 68 of the first strap 54. The force sensor 74 is configured to sense a force applied to the conduit securement device 50, produce a force signal representative of the force, determine when the force signal at least meets one or more thresholds and generate an alarm signal. The conduit securement device 50 further comprises an alarm (not shown) in communication with the force sensor and configured to receive the alarm signal and issue an alarm.

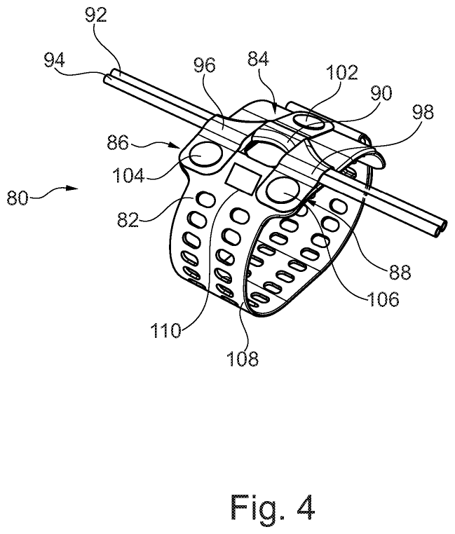

FIG. 4 shows a conduit securement device 80 comprising a support portion 82, an elastically extendable first strap 84 and two elastically extendable second straps 86, 88.

A portion 90 of the first strap 84 is configured to attach around a first region of conduits 92, 94. A portion 96, 98 of each of the second straps 86, 88 is adapted to attach to longitudinally adjacent second and third regions of the conduits 92, 94.

When the first strap 84 and the second straps 86, 88 are attached around the longitudinally adjacent first, second and third regions of the flexible conduits 92, 94, the conduits 92, 94 are urged into a curved or convoluted pathway, as shown. It will be understood that in alternative embodiments a conduit securement device may comprise a different number of straps and the conduit may be urged into alternative curved or convoluted pathways.

The first and second straps 84, 86, 88 are formed from an elastomeric material, and are each releasably attached at respective free ends to the support portion 82 of the device 80. Releasable attachment is effected by way of apertures through the free ends through which buttons 102, 104, 106 are introduced. The buttons and apertures together form releasable fixings. It will be understood that alternative embodiments of the fixings may be used.

The portion 90 of the first strap 84 is wrapped around the first region of the conduits 92, 94. The portions 96, 98 of the second straps 86, 88 are wrapped around the second and third regions of the conduits 92, 94 in an opposite sense. The portions 90, 96, 98 each define channels, which, in the embodiment shown, extend generally continuously from one another, together defining, at least in part, the curved or convoluted pathway of the conduits 92, 94.

The conduit securement device 80 is configured such that the portions 90, 96, 98 of the first and second straps 84, 86, 88 form channels that are laterally offset from one another (i.e. in a direction generally perpendicular to the direction of the channels). Accordingly, when the portions 90, 96, 98 of the first and second straps 84, 86, 88 are attached around the regions of the conduits 92, 94, the portions 90, 96, 98 are elastically extended. The first strap 84 applies a first force and the second straps 86, 88 each apply a generally opposed second force to the conduits 92, 94 to urge them into the curved/convoluted pathway.

When a force is applied between the conduit securement device 80 and the conduits 92, 94 proximate or distal to the securement device 80, at least a component of which acts longitudinally along the conduits 92, 94. This acts to try to straighten the convoluted pathway and further elastically extend the first and second straps 84, 86, 88. This increases the static friction between the straps and the conduits and resists longitudinal displacement of the conduits 92, 94 in relation to the securement device 80. Clinically sensitive areas, in particular the entry point of at least one of the conduits to a patient, can thereby be isolated from such forces applied to the conduits.

The inherent elasticity of the first and second straps 84, 86, 88 also enables the conduit securement device 80 to accommodate forces applied in other directions between the securement device 80 and the conduits 92, 94. In the embodiment shown, the portions 90, 96, 98 are not attached to the support portion 82 so are able to lift away therefrom and still further reduce the risk of folding or kinking the conduits 92, 94 where they extend out of the channels. In use with fluid line conduits, the elasticity of the straps 84, 86, 88 or the portions 90, 96, 98 of the straps, can be selected such that the straps will elastically extend or deform at lower forces than are required to compress the fluid line conduit.

The securement device 80 further includes an external attachment, in the embodiment shown an adjustable band 108, that extends from the support portion 82. This is used to attach the conduit securement device to a patient or a structure.

The curved or convoluted pathway of the conduits of aspects and embodiments disclosed herein isolates the conduits extending from either side of the securement device from forces applied to the conduit extending from the other side of the device. A symmetrical curved or convoluted pathway may be of further benefit in this regard. Accordingly, the securement device can be attached either way around, for example to either an upper or lower limb, torso, waist, head, neck of a patient.

The conduit securement device 80 comprises a force sensor 110 attached to a sensor portion of the device 80 comprising a central portion of the support portion 82. The force sensor 110 is configured to sense a force applied to the device 80 as a result of a force applied to the or each flexible conduit 92, 94, produce a force signal representative of the force, determine when the force signal meets one or more thresholds and generate an alarm signal. The conduit securement device 80 comprises an alarm (not shown) in communication with the force sensor 110 and configured to receive the alarm signal and issue an alarm.

The force sensors 26, 44, 74, 110 of the conduit securement devices 10, 30, 50, 80 can comprise any of a strain force sensor, a pressure force sensor, a dynamic force sensor, a rotation force sensor. The force sensors 26, 44, 74, 110 are configured to sense force applied to the devices 10, 30, 50, 80 as a result of a force applied to the conduits arising from any of pulling, bending, moving, vibrating, elongating the conduits.

The force sensors 26, 44, 74, 110 will produce a force signal representative of the force applied to the devices 10, 30, 50, 80 and process the force signal to quantify the force. The force sensors 26, 44, 74, 110 may further process the force signal to determine a direction of the force. The force sensors 26, 44, 74, 110 process the force signal to determine when the force signal at least meets a force magnitude threshold and at least meets a force duration threshold and generate an alarm signal. The force magnitude threshold may be any of 10 N, 20 N, 30 N, 40 N, 60 N, 100 N, 140 N. The force duration threshold may be any of 0.1 secs, 1 sec, 2 secs, 3 secs, 4 secs, 5 secs, 10 secs, 20 secs.

The force sensors 26, 44, 74, 110 each comprise a force sensing element and a signal processing unit in communication with the force sensing element. The force sensing elements sense a force applied to the conduit securement devices 10, 30, 50, 80, generate a force signal representative of the force and output the force signal to the signal processing units. The signal processing units determine when the force signal at least meets one or more thresholds and generates an alarm signal.

The force sensing elements are positioned at the sensor portions 22, 40, 68, 82 of the conduit securement devices 10, 30, 50, 80. The signal processing units may be co-positioned with the force sensing elements or may be positioned at a different location of the conduit securement devices. The force sensing elements may be in wireless or wired communication with the signal processing units. The force sensing elements may be attached to or moulded within the sensor portions 22, 40, 68, 82 of the conduit securement devices 10, 30, 50, 80.

The signal processing units each comprise at least one processor, configured to receive a force signal representative of a force applied to the conduit securement devices 10, 30, 50, 80 from the force sensing elements. The signal processing units each comprise at least one memory unit, configured to store force sensor data and one or more force magnitude thresholds and one or more force duration thresholds. The signal processing units each comprise at least one transceiver, which outputs force sensor data to one or more computing devices, such as a device located in proximity to a patient using a conduit securement device, a device located in a patient care facility or with one or more care personnel.

The force sensing elements may continuously monitor a force applied to the conduit securement devices 10, 30, 50, 80, as a result of a force applied to the conduit, and continuously produce a force signal representative of the force applied to the devices. The force sensing elements may continuously output the force signal to the signal processing units. The signal processing units may store the force signals, push or notify the force signals to one or more computing devices. The force signals may be continuously captured by the one or more computing devices. The force signals may be continuously displayed by the one or more computing devices.

The alarms of the conduit securement devices 10, 30, 50, 80 may comprise any of an audible alarm element, a visual alarm element, a haptic alarm element configured to receive the alarm signal and respectively issue an audible alarm, a visual alarm, a haptic alarm.

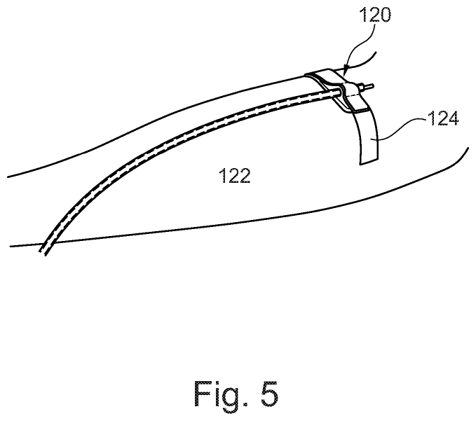

Referring to FIG. 5, an embodiment of a conduit securement device 120 is shown attached to an arm 122 of a patient. The conduit securement device 120 is of the type shown in FIGS. 2a and 2b. The conduit securement device 120 comprises an external attachment in the form of an adhesive strip 124 which attaches the device 120 to the arm 122 of the patient. It will be appreciated that the embodiments of the conduit securement device shown in FIGS. 1, 3 and 4 can also be attached to an arm of a patient.

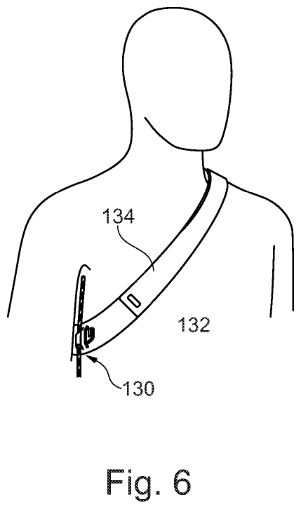

Referring to FIG. 6, an embodiment of a conduit securement device 130 is shown attached to a torso 132 of a patient. The conduit securement device 130 is of the type shown in FIG. 1. The conduit securement device 130 comprises an external attachment in the form of a band 134 which crosses over the torso 132 and around a shoulder of the patient, as shown, to attach the device 130 to the torso 132 of the patient. It will be appreciated that the embodiments of the conduit securement device shown in FIGS. 2, 3 and 4 can also be attached to the torso of a patient.

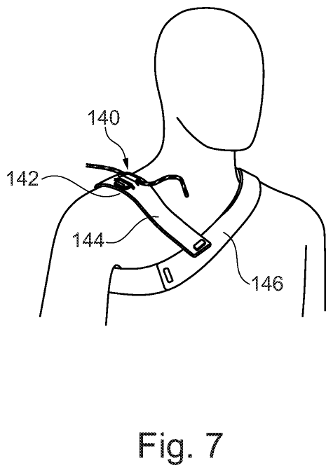

Referring to FIG. 7, an embodiment of a conduit securement device 140 is shown attached to a shoulder 142 of a patient. The conduit securement device 140 is of the type shown in FIG. 1. The conduit securement device 140 comprises an external attachment in the form of a first band 144 and a second band 146 which cross over the shoulders and under an arm of the patient, as shown, to attach the device 140 to the shoulder 142 of the patient. It will be appreciated that the embodiments of the conduit securement device shown in FIGS. 2, 3 and 4 can also be attached to a shoulder of a patient.

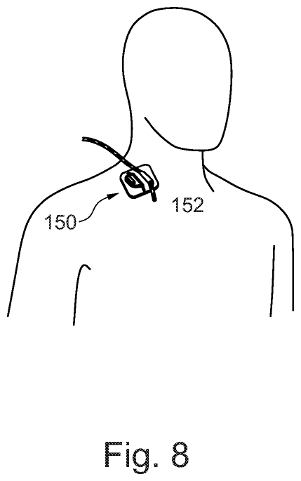

Referring to FIG. 8, an embodiment of a conduit securement device 150 is shown attached to the neck 152 of a patient. The conduit securement device 150 is of the type shown in FIG. 1. The conduit securement device 150 comprises an external attachment in the form of an adhesive strip (not shown) beneath the device 150 which attaches the device 150 to the neck 152 of the patient. It will be appreciated that the embodiments of the conduit securement device shown in FIGS. 2, 3 and 4 can also be attached to the neck of a patient.

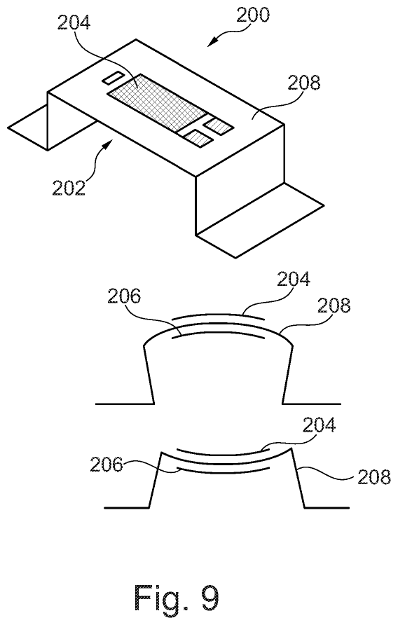

Referring to FIG. 9, a force sensor comprising a strain sensor 200 is shown, which is configured to sense a strain force applied to a conduit securement device, produce a strain signal representative of the strain force, determine when the strain signal at least meets one or more thresholds and generate an alarm signal.

The strain sensor 200 processes the strain signal to determine when the strain signal at least meets a magnitude strain threshold and generates an alarm signal. The strain sensor may also process the strain signal to determine when the strain signal at least meets a magnitude strain threshold and at least meets a duration threshold and generate an alarm signal. When the conduits being secured are, for example, fluid lines for a patient, such a duration of force on the conduit securement device resulting from a force on the conduits may result in dislodgement of one or more of the fluid line conduits from the patient.

The strain sensor 200 comprises a strain sensing element 202 and a signal processing unit (not shown) in communication with the strain sensing element 202. The strain sensing element 202 senses a strain applied to a conduit securement device, generates a strain signal representative of the strain and outputs the strain signal to the signal processing unit. The signal processing unit determines when the strain signal at least meets one or more thresholds and generate an alarm signal.

The strain sensing element 202 comprises a first strain gauge 204 and a second strain gauge 206. The strain gauges 204, 206 are placed on opposite sides of a base 208 of the strain sensor 200. The base 208 is made of a sheet metal material, hardened to retain its bridge-like shape. The base 208 may provide strain relief to protect the strain gauges 204, 206.

The strain gauges 204, 206 comprise a strain-sensitive material which senses a strain force by changing resistance of the material on flexing of the material. The strain-sensitive material may, for example, comprise a carbon-impregnated polyethylene strain-sensitive material. The carbon-impregnated polyethylene strain-sensitive material may comprise a Velostat® strain-sensitive material. The Velostat® strain-sensitive material may sense a strain force by changing resistance of the material.

The strain sensing element 202 is attached to a sensor portion of a conduit securement device comprising a central section of the support portion of the device. This central section of the device visibly stretches on pulling of a conduit secured to the device. This causes stretching of the strain gauges 204, 206, which changes the resistance of the gauges. This is detected as a strain force applied to the conduit securement device, and a strain signal representative of the strain force is produced.

The signal processing unit may be co-located with the strain sensing element 202 and contained within a housing. The housing further contains the alarm (not shown). The housing also contains a power source (not shown) for the strain sensing element 202, the signal processing unit and the alarm.

The signal processing unit comprises an interface for the strain sensing element 202, which may comprise a Wheatstone bridge (not shown). The Wheatstone bridge may be connected to an ADC (not shown). This may be a dual channel, 24 bit ADC with programmable gain etc.

The ADC is connected to a control unit (not shown), such as a multi-point control unit, for example, an Arduino Teensy 2.0 control unit, having the required inputs, outputs, power supply connector, etc. The signal processing unit 208 may be of a size of, for example, approximately 30 mm×25 mm. The power supply is a coin cell battery (not shown), for example a CR2032 coin cell battery.

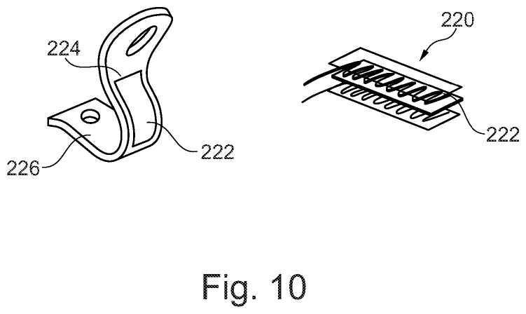

Referring to FIG. 10, a force sensor comprising a pressure sensor 220 is shown, which comprises a pressure sensing element 222 moulded within a sensor portion 224 of a first strap 226 of a conduit securement device and a signal processing unit (not shown) attached to a support portion of the device. The pressure sensing element 222 comprises a strip of pressure-sensitive material which senses the pressure force by a change of resistance of the material, in this embodiment, conductive fabric pressure-sensitive material.

The sensor portion 224 of the first strap 226 which is configured to loop around a region of a conduit when secured to the device. When a force is applied to the conduit, for example by pulling the conduit, the first strap 226 elastically extends, or further elastically extends which applies a pressure force to the first strap 226 and to the pressure sensing element 222 attached to the first strap 226. The pressure sensing element 222 senses the pressure force and generates a pressure signal representative of the pressure force and outputs the pressure signal to the signal processing unit. The signal processing unit is wirelessly connected to the pressure sensing element 222. The signal processing unit comprises at least one processor (not shown) which determines when the pressure signal at least meets one or more thresholds and generates an alarm signal.

The signal processing unit of the pressure sensor 220 may process the pressure signal to determine when the pressure signal at least meets a magnitude pressure threshold and generate an alarm signal. The signal processing unit may process the pressure signal to determine when the pressure signal at least meets a magnitude pressure threshold and at least meets a duration threshold and generate an alarm signal.

The signal processing unit sends a signal to an alarm (not shown) in communication with the pressure sensor 220 and configured to receive the alarm signal and issue an alarm. The alarm may be placed within the signal processing unit.

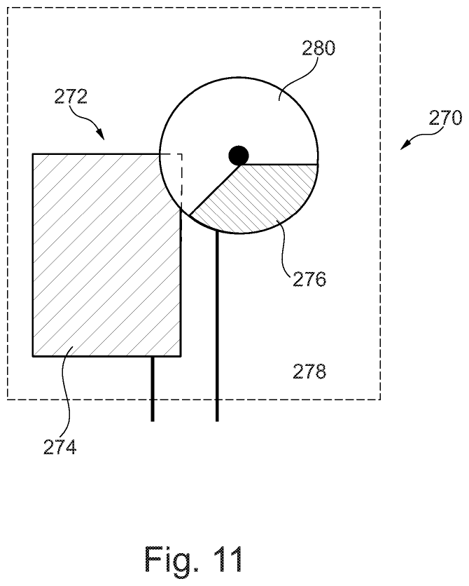

Referring to FIG. 11, a force sensor comprising a rotation sensor 270 is shown, which is configured to sense a rotation force applied to a first strap of a conduit securement device, produce a rotation signal representative of the rotation force, determine when the rotation signal at least meets one or more thresholds and generate an alarm signal.

The rotation sensor 270 processes the rotation signal to determine when the rotation signal at least meets a magnitude rotation threshold and generates an alarm signal. The strain sensor may also process the rotation signal to determine when the rotation signal at least meets a magnitude rotation threshold and at least meets a duration threshold and generate an alarm signal.

The rotation sensor 270 comprises a rotation sensing element 272 and a signal processing unit (not shown) in communication with the rotation sensing element 272. The rotation sensing element 272 senses a rotation applied to a first strap of a conduit securement device, generates a rotation signal representative of the rotation and outputs the rotation signal to the signal processing unit. The signal processing unit determines when the rotation signal at least meets one or more thresholds and generate an alarm signal.

The rotation sensing element 272 comprises a first plate 274 and a second plate 276. The first plate 274 is placed on a support portion 278 of the conduit securement device. The second plate 276 is attached to the first strap 280 of the device. The first and second plates 274, 276 are made of a conductive sheet material and together form a capacitor. As the conduit is pulled, the first strap 280 rotates, changing the amount of overlapping area between the capacitor plates 274, 276, which produces a rotation signal representative of the rotation force on the strap 280.

The signal processing unit may be co-located with the rotation sensing element 272 and contained within a housing. The housing may further contains an alarm (not shown). The housing may also contain a power source (not shown) for the rotation sensing element 272, the signal processing unit and the alarm.

Whilst the invention has been described in connection with the foregoing illustrative embodiments, various modifications, additions and alterations may be made to the invention by one skilled in the art without departing from the scope of the claims.

1. A conduit securement device comprising:
a support portion,
at least one elastically extendable first strap coupled to the support portion and comprising a portion configured to attach around a first region of a conduit,
at least one force sensor attached to a sensor portion of the device and configured to sense a force applied to the device as a result of a force applied to the conduit, produce a force signal representative of the force applied to the device, determine when the force signal meets one or more thresholds and generate an alarm signal, and
an alarm in communication with the force sensor and configured to receive the alarm signal and issue an alarm.

2. A conduit securement device according to claim 1 in which the force sensor comprises one or more of a strain force sensor, a pressure force sensor, a dynamic force sensor, a rotation force sensor.

3. A conduit securement device according to claim 1 in which the force sensor processes the force signal to determine when the force signal at least meets a magnitude threshold and generates an alarm signal; or in which the force sensor processes the force signal to determine when the force signal at least meets both a magnitude threshold and a duration threshold and generates an alarm signal.

4. (canceled)

5. A conduit securement device according to claim 1 in which the force sensor comprises a force sensing element and a signal processing unit in communication with the force sensing element, the force sensing element senses a force applied to the conduit securement device, generates a force signal representative of the force and outputs the force signal to the signal processing unit and the signal processing unit determines when the force signal at least meets one or more thresholds and generate an alarm signal.

6. A conduit securement device according to claim 5 in which the signal processing unit comprises at least one memory unit configured to store force sensor data comprising any of one or more magnitudes of one or more force signals representative of forces applied to the conduit securement device, one or more instances at which a force signal at least meets at least one threshold, one or more instances at which an alarm signal is generated, magnitudes of force signals which at least meet at least one threshold, duration of force signals which at least meet at least one threshold, one or more force magnitude thresholds, one or more force duration thresholds.

7. (canceled)

8. A conduit securement device according to claim 1 in which the force sensor comprises a strain sensor configured to sense a strain force applied to the conduit securement device produce a strain signal representative of the strain force, determine when the strain signal at least meets one or more thresholds and generate an alarm signal.

9. A conduit securement device according to claim 8 in which the strain sensor comprises a strain sensing element and a signal processing unit and the strain sensing element comprises any of a strain gauge, a strain-sensitive material.

10. A conduit securement device according to claim 9 in which the strain sensing element is attached to a sensor portion of the conduit securement device comprising a central section of the support portion of the device.

11. A conduit securement device according to claim 1 in which the force sensor comprises a pressure sensor configured to sense a pressure force applied to the conduit securement device, produce a pressure signal representative of the pressure force, determine when the pressure signal at least meets one or more thresholds and generate an alarm signal.

12. A conduit securement device according to claim 11 in which the pressure sensor comprises a pressure sensing element which comprises a pressure-sensitive material which senses a pressure force by changing electrical state of the material.

13. A conduit securement device according to claim 12 in which the pressure sensing element is attached to a sensor portion of the conduit securement device comprising a portion of the first strap configured to attach around a first region of a conduit; or in which the pressure sensing element is attached to a sensor portion of the conduit securement device comprising a portion of one or more of the second straps configured to attach around a region of a conduit.

14. (canceled)

15. A conduit securement device according to claim 1 in which the force sensor comprises a rotation force sensor to sense a rotation caused by a force applied to one or more parts of the conduit securement device, such as the first strap, when the conduit is pulled.

16. A conduit securement device according to claim 1 in which the force sensor comprises a dynamic force sensor configured to sense a dynamic force applied to the conduit securement device, produce a dynamic force signal representative of the dynamic force, determine when the dynamic force signal at least meets one or more thresholds and generate an alarm signal.

17. A conduit securement device according to claim 16 in which the dynamic force sensor comprises a dynamic force sensing element comprising a piezoelectric material.

18. A conduit securement device according to claim 17 in which the dynamic force sensing element is attached to a sensor portion of the conduit securement device comprising a portion of one or more of the second straps configured to attach around a region of a conduit.

19. A conduit securement device according to claim 1 in which the force sensor comprises a stretch sensor comprising any of a capacitive stretch sensor, a resistive stretch sensor.

20. A conduit securement device according to claim 19 in which the stretch sensor comprises a conductive material attached to or printed on or impregnated into a section of the conduit securement device.

21. (canceled)

22. A conduit securement device according to claim 1 comprising an accelerometer to measure general movement of a patient wearing the conduit securement device.

23. A conduit securement device according to claim 22 in which one or more instances at which an alarm signal is generated are correlated with one or more measurements from the accelerometer to identify patient activity at the one or more instances to determine causes of the alarms.

24. (canceled)

25. A medical system comprising a conduit securement device according to claim 1 and at least one conduit secured to the device.
